(19) United States
(12) Patent Application Publication
(10) Pub. No.: US 2002/0019336 A1
(43) Pub. Date: Feb. 14, 2002

(54) Sustained release drug compositions

(76) Inventors: Aki Kitagawa, Kanagawa (JP); Yutaka Mizushima, Tokyo (JP); Yukie Takagi, Kanagawa (JP); Rie Igarashi, Kanagawa (JP)

Correspondence Address: Y. ROCKY TSAO, Fish & Richardson PC, 225 Franklin Street, Boston, MA, 02110-2804, US

(21) Appl. No.: 09/834,103

(22) Filed: Apr. 12, 2001

(30) Foreign Application Priority Data
Apr. 17, 2000 (JP) 2000-115091
Jul. 5, 2000 (JP) 2000-203850

(51) Int. Cl. A61K 38/28; A61K 31/715; A61K 38/20; A61K 38/21; A61K 39/395

(52) U.S. Cl. 514/2; 424/85.2; 514/3; 514/54; 424/130.1; 424/85.5; 424/85.6

(57) ABSTRACT

The invention relates to a composition providing sustained release of a drug, the composition including (1) a mucopolysaccharide, a carrier protein, and a drug; or (2) a mucopolysaccharide and a protein drug.

### CROSS-REFERENCE TO RELATED APPLICATIONS

This application claims priority from Japanese Patent Application Nos. 2000-115091, filed Apr. 17, 2000 and 2000-203850, filed Jul. 5, 2000.

### BACKGROUND OF THE INVENTION

Drug compositions that facilitate even, sustained release of a drug after administration to a subject are beneficial for a variety of reasons. For example, if the drug is an antigen and the composition is a vaccine, sustained release of the antigen can result in a more vigorous immune response or stimulate memory immunity. In addition, many drugs may not be effective, and can perhaps be dangerous, if released in a burst rather than gradually through time.

Unfortunately, currently available sustained release drug compositions are characterized by a number of disadvantages. Some so-called sustained release compositions will release substantially all of a drug in a composition within 24 hours, even though the ideal regimen, such as for a vaccine, requires sustained release for at least a few days. Other compositions involve chemically treated or cross-linked polymers. Since the chemical treatment often necessitates the use of toxic chemicals, the resulting drug composition containing such polymers require safety validation before use. Even without chemical treatment, still other sustained release drug compositions require laborious, intricate, or complex production methods, thereby increasing the costs of sustained release drug compositions.

### SUMMARY OF THE INVENTION

The invention is based on the discovery that a sustained release drug composition can be produced by mixing a drug with a mucopolysaccharide and optionally a carrier protein. These drug compositions are easy and inexpensive to produce, while delivering long lasting sustained release of a drug.

Accordingly, the invention features a composition providing sustained release of a drug, the composition including a mucopolysaccharide (e.g., chondroitin sulfate or hyaluronate), a carrier protein (e.g., γ-globulin, albumin, fibrinogen, histone, protamine, gelatin, or collagen), and a drug (e.g., a protein drug). In some embodiments, the composition can include only the mucopolysaccharide, the carrier protein, the drug, and one or more pharmaceutically acceptable additives. The ratio of the total mass of mucopolysaccharide in the composition to the total mass of carrier protein in the composition can be about 1:1 to 1:20. Further, the composition can contain about 0.1 to 50% by weight (e.g., about 1% to 40%, 5% to 30%, or 10% to 15% by weight) the mucopolysaccharide or about 0.1 to 2% by weight (e.g., about 0.5% to 1% by weight) the drug.

Examples of protein drugs that can be included in the compositions of the invention are erythropoietin, granulocyte colony stimulating factor, granulocyte macrophage colony stimulating factor, thrombopoietin, interferon-α, interferon-β, interferon-γ, urokinase, tissue plasminogen activator, interleukin-11, fibroblast growth factor, epidermal growth factor, growth hormone, brain-derived neurotrophic factor, nerve growth factor, leptin, neurotrophin-3, superoxide dismutase, antibody, calcitonin, insulin, and parathyroid hormone.

The invention further includes a method of producing any of the sustained release drug compositions of the invention by providing a precipitating solution containing a mucopolysaccharide, a carrier protein, and a drug; lowering the pH of the precipitating solution to a level sufficient to form an insoluble product containing the mucopolysaccharide, the carrier protein, and the drug; and collecting from the precipitating solution the insoluble product. In some embodiments, the insoluble product includes only the mucopolysaccharide, the carrier protein, the drug, and one or more pharmaceutically acceptable additives. The pH of the solution can be about 7 or above before the lowering step, and the pH of the solution can be lowered to about 2 to 4 (e.g., about 3) in the lowering step. In addition, the method can include, prior to the providing step, mixing a first solution containing the carrier protein and the drug with a second solution containing the mucopolysaccharide to produce the precipitating solution. To facilitate precipitation, the precipitating solution can contain zinc or calcium ions. In other embodiments, the method further includes suspending the insoluble product in a preparatory solution having a pH of about 6 to 8 to form a mixture; and lyophilizing the mixture to obtain a solid product.

In another aspect, the invention features a composition providing sustained release of a drug, the composition including a mucopolysaccharide (e.g., chondroitin sulfate or hyaluronate) and a protein drug (e.g., as described herein). In some embodiments, the composition contains only the mucopolysaccharide, the protein drug, and one or more pharmaceutically acceptable additives. The composition can contain about 0.1 to 50% by weight (e.g., about 1% to 40%, 5% to 30%, or 10% to 15% by weight) the mucopolysaccharide or about 0.1 to 50% (e.g., about 0.5% to 1% by weight) by weight the protein drug.

The invention further includes a method of producing any of the sustained release drug composition of the invention by providing a precipitating solution containing a mucopolysaccharide and a protein drug; lowering the pH of the precipitating solution to a level sufficient to form an insoluble product containing the mucopolysaccharide and the protein drug; and collecting from the precipitating solution the insoluble product. The pH of the solution can be about 7 or above before the lowering step, and the pH of the solution can be lowered to about 2 to 4 (e.g., about 3) in the lowering step. The method further includes, prior to the providing step, mixing a first solution containing the protein drug with a second solution containing the mucopolysaccharide to produce the precipitating solution. The precipitating solution can contain zinc or calcium ions to facilitate precipitation. In some embodiments, the method further includes suspending the insoluble product in a preparatory solution having a pH of about 6 to 8 to form a mixture; and lyophilizing the mixture to obtain a solid product.

The invention also includes a method of delivering a drug to a subject (e.g., a human) by introducing (e.g., subcutaneously or intramuscularly) a sustained release composition of the invention into the subject.

A “mucopolysaccharide” is a polysaccharide containing repeating units (e.g., disaccharide units) of uronic acid (e.g., glucuronic acid) or galactose and hexoseamine (e.g., N-acetylglucosamine and N-acetylgalactoseamine).

A “carrier protein” is any protein having a primary role within a sustained release drug composition that is not related to a biological activity. Rather, a carrier protein's main role is to facilitate the binding of the drug to other components of the composition, such as the mucopolysaccharide. Particularly useful carrier proteins include globulins (e.g., human γ-globulin) and albumins (e.g., human serum albumin).

The methods and compositions of the invention are particularly useful for formulating sustained release drug compositions when the drug is itself a protein or has a high binding affinity for a carrier protein. However, even if the drug is a small molecule drug that does not bind protein well, a sustained release drug composition can still be produced, e.g., by using precipitating agents such as zinc or calcium in the precipitating solution. In addition, the compositions maintain the biological activity associated with the drug, it is believed in part because the preparation of the sustained release composition does not utilize harsh chemicals nor involve extreme physical conditions.

Other features or advantages of the present invention will be apparent from the following detailed description, and also from the claims.

### BRIEF DESCRIPTION OF THE DRAWINGS

FIG. 1 is a line graph showing sustained release of radiolabeled protein from a composition prepared in accordance with the invention. The composition contains sodium chondroitin sulfate and human γ-globulin, with indicated ratios of the respective components.

FIGS. 2 and 3 are line graphs showing sustained release of radiolabeled human γ-globulin from a composition containing a 1:2 weight ratio of sodium chondroitin sulfate (MW 230,000) to human γ-globulin.

FIGS. 4 and 5 are line graphs showing sustained release of albumin from a composition containing a 1:2 weight ratio of sodium chondroitin sulfate and human serum albumin.

FIG. 6 is a line graph showing the release of lecithinized superoxide dismutase (PC-SOD) after administration of a sustained release composition containing the PC-SOD was administered to mice. PC-SOD alone was administered as a control.

FIG. 7 is a line graph showing the release of indomethacin from a sustained release composition of the invention.

FIG. 8 is a line graph showing in vivo release of radiolabeled human γ-globulin after subcutaneous administration of γ-globulin alone or as part of a sustained release composition. Radioactivity below 500 cpm was defined as background.

FIG. 9 is a line graph showing release of basic fibroblast growth factor (bFGF) from a sustained release composition containing 1% by weight human serum albumin.

### DETAILED DESCRIPTION

The invention relates to inexpensive and easy to produce sustained release drug compositions that maintain the biological activity of the drugs. This result is accomplished by mixing a mucopolysaccharide (e.g., a naturally occurring mucopolysaccharide) with either (1) a protein drug or (2) a carrier protein plus a non-protein drug in a neutral or basic pH. It is noted, however, that a carrier protein can be added to the composition, even if the drug itself is a protein, to facilitate precipitation or binding to the mucopolysaccharide. The pH of the resulting mixture is then lowered to a pH sufficient to form an insoluble material containing the drug and the mucopolysaccharide. Contemplated within the scope of this invention is a vaccine composition containing a sustained release composition including an antigen as a biologically active ingredient.

The compositions of the present invention can be administered via any appropriate route, e.g. intravenously, intraarterially, topically, by injection, intraperitoneally, intrapleurally, subcutaneously, intramuscularly, sublingually, intraepidermally, or rectally.

Any sustained release composition of the invention can contain one or more pharmaceutically acceptable additives. It can be formulated as a suspension, suppository, tablet, granules, powder, capsules, ointment, or cream. In the preparation of these pharmaceuticals, a solvent (e.g., water or physiological saline), solubilizing agent (e.g., ethanol, Polysorbates, or Cremophor EL), agent for making isotonicity, preservative, antioxidizing agent, excipient (e.g., lactose, starch, crystalline cellulose, mannitol, maltose, calcium hydrogen phosphate, light silicic acid anhydride, or calcium carbonate), binder (e.g., starch, polyvinylpyrrolidone, hydroxypropyl cellulose, ethyl cellulose, carboxy methyl cellulose, or gum arabic), lubricant (e.g., magnesium stearate, talc, or hardened oils), or stabilizer (e.g., glucose, lactose, mannitol, maltose, polysorbates, macrogols, or polyoxyethylene hardened castor oils) can be added. If appropriate, glycerin, dimethylacetamide, 70% sodium lactate, a surfactant, or a basic substance such as sodium hydroxide, ethylenediamine, ethanolamine, sodium bicarbonate, arginine, meglumine, or trisaminomethane is added. Pharmaceutical preparations such as solutions, tablets, granules or capsules can be formed with these pharmaceutically acceptable additives.

The dose of the compound of the present invention is determined in consideration of the results of animal experiments and various conditions. More specific doses vary depending on the administration method, the condition of the subject such as age, body weight, sex, sensitivity, food eaten, dosage intervals, medicines administered in combination, and the source, seriousness, and degree of pain. The optimal dose and the administration frequency under a given condition must be determined by the appropriate dosage test of a medical specialist based on the aforementioned guide.

In a typical in vitro evaluation test, a carrier protein (e.g., human γ-globulin, human serum albumin, or fibrinogen) and an acid mucopolysaccharide (e.g., sodium chondroitin sulfate or sodium hyaluronate) are mixed in respective weight ratios of 4:1, 3:1, 2:1, 1:1, and 1:2), with the concentration of mucopolysaccharide being fixed at 1% of composition weight. The pH of the precipitating solution is lowered to about pH 3, and an insoluble product is obtained by, e.g., centrifugation. The harvested insoluble product is then suspended in phosphate buffered saline (PBS) for a timed release test. At pre-determined times after the product is suspended in the buffer, the reaction is centrifuged, and a portion of the supernatant is tested for release of the drug. The reaction is then agitated, incubated at 37° C., and then tested again at the next pre-determined time point.

In a typical in vivo evaluation test, the sustained release composition is subcutaneously injected as one bolus into mice, though additional boli can be used. After injection or implantation, blood samples can be collected, at pre-determined time points, from the mice and assayed for amount of drug or test compound originally present in the composition. Alternatively, the composition can be locally applied, e.g., topically to a skin lesion. A biopsy at pre-determined distance from the local application site can be obtained at a pre-determined time points. The presence and amount of drug or test compound originally present in the composition is then assayed in each biopsy sample.

Without further elaboration, it is believed that one skilled in the art can, based on the above disclosure and the examples below, utilize the present invention to its fullest extent. The following examples are to be construed as merely illustrative of how one skilled in the art can make and use the present sustained release compositions, and are not limitative of the remainder of the disclosure in any way. All publications and references cited in this disclosure are hereby incorporated by reference.

### EXAMPLE 1

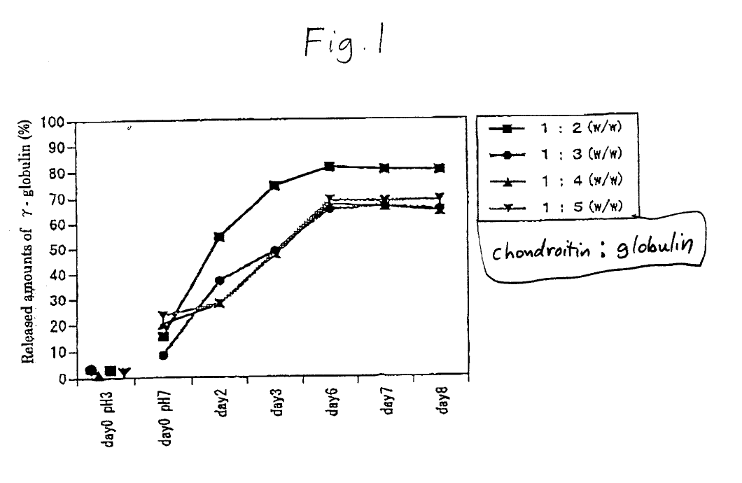

In a preliminary evaluation test, human γ-globulin and sodium chondroitin sulfate were mixed in respective weight ratios of 4:1, 3:1, 2:1, 1:1, and 1:2, with the concentration of the chondroitin being fixed at 1% of composition weight. The pH of the precipitating solution was lowered to about pH 3, and an insoluble product was obtained by centrifugation. The harvested insoluble product was then suspended in PBS for a timed release test. At pre-determined times after the product was suspended in the buffer, the reaction was centrifuged, and a portion of the supernatant was tested for release of the drug. The mixture was then agitated, incubated at 37° C., and then tested again at the next pre-determined time point. The results, as shown in FIG. 1, indicated that compositions with ratios of about 1:2 and 1:3 provided release of more drug than other ratios.

### EXAMPLE 2

Thirty microliters of a 100 mg/ml solution of sodium chondroitin sulfate A (Sigma, MW 4-50×10⁶) was mixed with 200 μl of a 30 mg/ml solution of human γ-globulin and 370 μl of PBS. The weight ratio of chondroitin to globulin was therefore about 1:2, and the volume of the resulting mixture was about 600 μl.

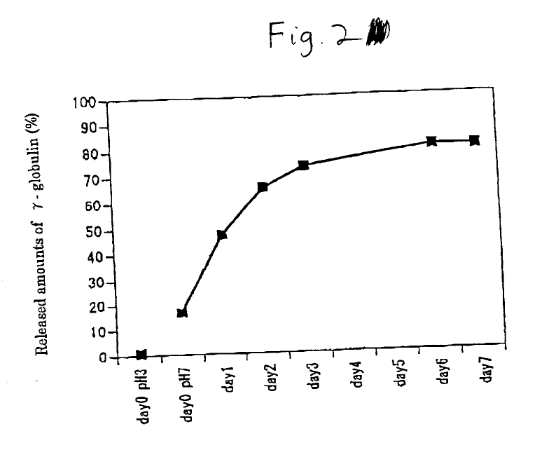

Fifty microliters of 0.2 N HCl was gently added to the mixture to adjust the pH to about 3. The reaction was then mixed using a vortex mixer and centrifuged at 3000 rpm for five minutes. The supernatant was removed and replaced with 1 ml of PBS. A portion of this initial supernatant was tested for protein content. Thereafter, at specified time points, the reaction was centrifuged at 3000 rpm for five minutes, and a 25 μl portion of the supernatant was removed and assayed for protein content. During the test period, the reaction was incubated at 37° C. Protein content of the samples was measured using the Lowry method (kit from BioRad), with a human γ-globin standard curve being generated using human γ-globin purchased from Sigma. The results, shown in FIG. 2, indicate a release rate of about 30%/day for the first two days and 5%/day until the seventh day. Achieving sustained release for this length of time has been difficult in the past, and therefore this result was unexpectedly superior to known sustained release drug compositions.

### EXAMPLE 3

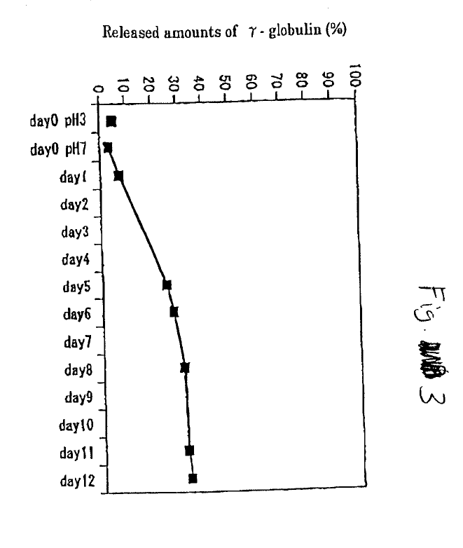

One hundred and fifty microliters of a 20 mg/ml solution of sodium hyaluronate (Seikagaku Kogyo, MW about 23×10⁵) was mixed with 200 μl of a 30 mg/ml solution of human γ-globulin and 250 μl of PBS in a microfuge tube. The weight ratio of hyaluronate to globulin was therefore about 1:2, and the total volume of the mixture was about 600 μl. Fifty microliters of 0.2 N HCl was gently added to the mixture to adjust the pH to about 3. The reaction was then mixed using a vortex mixer and centrifuged at 10,000 rpm for five minutes. The supernatant was removed and replaced with 1 ml of PBS. A portion of this initial supernatant was tested for protein content. Thereafter, at specified time points, the reaction was centrifuged at 10,000 rpm for five minutes, and a 25 μl portion of the supernatant was removed and assayed for protein content. Testing was performed as indicated in Example 2, except that centrifugation was performed at 10,000 rpm. The results, shown in FIG. 3, indicate a release rate of about 4%/day for the time period tested. Again, achieving sustained release for this length of time has been difficult in the past, and therefore this result was unexpectedly superior to known sustained release drug compositions.

### EXAMPLE 4

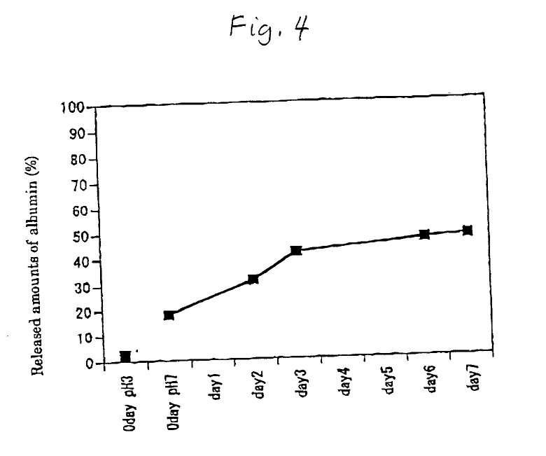

Thirty microliters of a 100 mg/ml solution of sodium chondroitin sulfate A (Sigma, MW about 4-50×10⁶) was mixed with 200 μl of a 30 mg/ml solution of human serum albumin and 370 μl of PBS in a microfuge tube. The weight ratio of chondroitin to albumin was therefore about 1:2, and the total volume fo the mixture was about 600 μl. The reaction was then treated and tested as described in Example 2, except that the protein standard for quantitation was human serum albumin purchased from Sigma. The results, shown in FIG. 4, indicate a release rate of about 5%/day for the time period tested.

### EXAMPLE 5

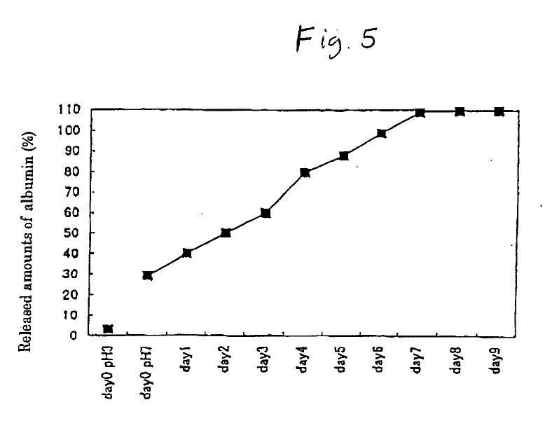

The experiment of Example 3 was repeated, but the human γ-globulin was replaced with human serum albumin (Sigma). The results are shown in FIG. 5.

### EXAMPLE 6

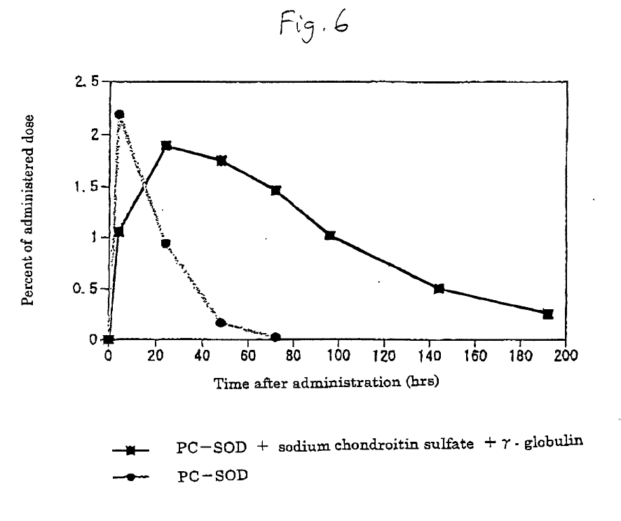

A sustained release preparation containing [³H]-lecithinized superoxide dismutase (PC-SOD), sodium chondroitin sulfate, and human γ-globulin was prepared. Two hundred microliters of a 10 mg/ml solution of sodium chondroitin sulfate A was mixed with [³H]-lecithinized superoxide dismutase (10 μCi, 6 mg as SOD), and adjusted to pH 3 with 0.1 N HCl. The resulting insoluble product was subcutaneously injected into mice as a single bolus at the back of the C3H mice. Control mice received ³H-labeled PC-SOD only. Blood was collected from inferior ophthalmic vein of the mice and assayed for radioactivity as a measure of PC-SOD released into the systemic circulation of the mice. The results, shown in FIG. 6, indicate that sustained release of PC-SOD was accomplished using a composition of the invention.

### EXAMPLE 7

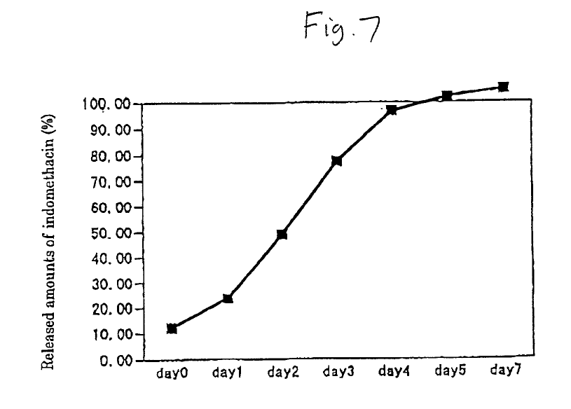

Sixty microliters of a 5 mg/ml indomethacin solution in ethanol (Wako Pure Chemicals) was mixed with 5 μl of [¹⁴C]-indomethacin in ethanol (100 μCi/ml, Daiichi Chemicals). To this mixture, 100 μl of a 30 mg/ml solution of human serum albumin (Sigma) and 200 μl of a 30 mg/ml solution of human γ-globulin (Sigma) were added. Finally, 30 μl of a 100 mg/ml solution of chondroitin sulfate and 805 μl of PBS were added. The weight ratio of chondroitin to total protein in this final mixture was therefore about 1:3, and the total volume was about 1.2 ml. One hundred microliters of 0.2 N HCl was gently added to the mixture to adjust the pH to about 3. The mixture was then treated and tested as described in Example 2, except that indomethacin was quantitated by mixing 100 μl of supernatant with 5 ml of scintillation cocktail (Packard), and then counting the radioactivity as a measure of indomethacin content. The results are shown in FIG. 7 and indicate that the present invention is applicable to small molecule drugs such as indomethacin.

### EXAMPLE 8

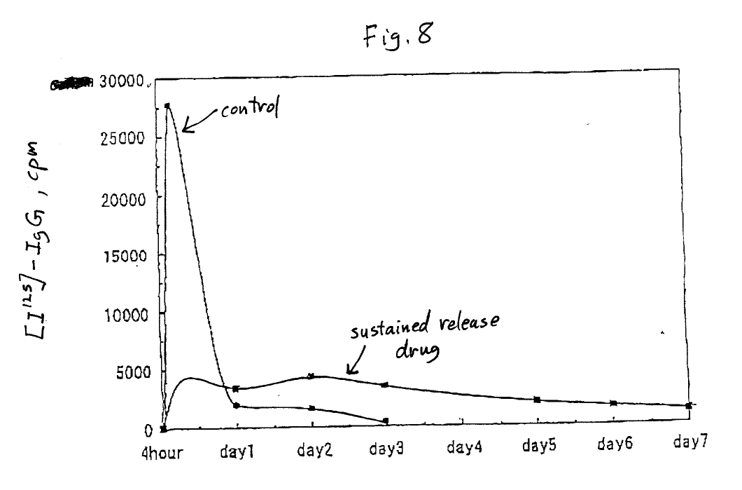

One hundred and fifty microliters of a 20 mg/ml solution of human γ-globulin (Sigma) was mixed with 10 μCi [¹²⁵I]-human IgG (ICN Biochemicals). To this solution, 100 μl of a 10 mg/ml sodium chondroitin sulfate solution and 100 μl of PBS was added, and the mixture mixed well. The weight ratio of chondroitin to total protein was therefore about 1:3. To the mixture, 100 μl of 0.2 N HCl was added to achieve a pH of about 3. The mixture was mixed using a vortex mixer and then centrifuged at 1500 rpm for 10 minutes at 4° C. The initial supernatant was replaced with 450 μl of PBS, and a portion of the supernatant was tested for IgG content. The insoluble product was suspended in 450 μl PBS and then subcutaneously injected at the back of the C3H mice (about 3 weeks of age). Fifty microliters of blood was collected from the fundus oculi of the mice at specified times after administration of the insoluble product. A mixture of globulin and [¹²⁵I]-human IgG, without a mucopolysaccharide, was used as the control. The results, shown in FIG. 8, indicate that a sustained release of globulin was achieved for at least several days subsequent to when release of control globulin could no longer be detected.

### EXAMPLE 9

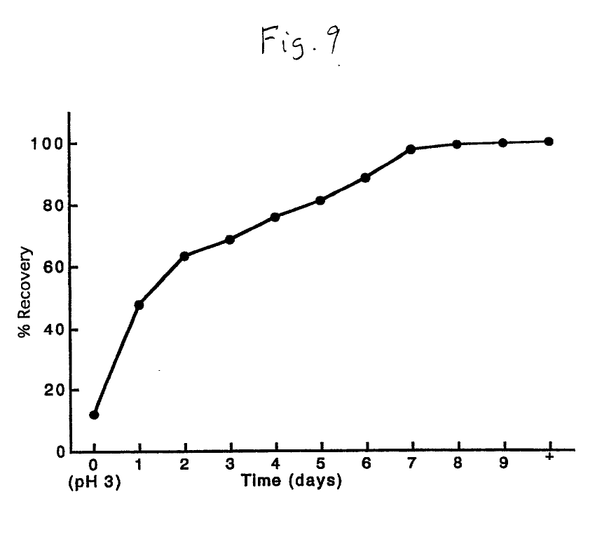

Ten microliters of a 100 μg/ml solution of basic fibroblast growth factor (bFGF) and 450 μl of PBS containing 9 mg of human γ-globulin were mixed in a microfuge tube. Then 300 μl of a 1% sodium chondroitin sulfate A solution in PBS was added, and the mixture was well stirred. To the mixture, hydrochloric acid (0.2 N) was gently added to adjust the pH to about 3. The reaction was and then centrifuged at 1000 rpm for 10 minutes. The supernatant was replaced with 1 ml of PBS containing 1% human serum albumin, and a small portion of the supernatant was assayed for bFGF content. The mixture containing the insoluble product was centrifuged at 1000 rpm for 10 minutes. At pre-determined time points, 25 μl of the supernatant was removed and assayed for bFGF content. During the period of observation, the insoluble produce was incubated at about 28° C. bFGF was measured using a Quantikine Human bFGF ELISA Kit (R&D System, Inc. MN, USA). As shown in FIG. 9, sustained release of bFGF was achieved.

### EXAMPLE 10

The compositions listed in Table 1 below were generated essentially according to Example 9, with modifications as indicated.

TABLE 1

| | | | Sodium | |
|---|---|---|---|---|
| | | Human γ- | chondroitin | Precipitation |
| Test materials | bFGF | globulin | sulfate | at pH 3 |
| bFGF pellet | 1 μg | 100 μl (1 mg) | 300 μl (3 mg) | yes |
| bFGF suspension | 1 μg | 100 μl (1 mg) | 300 μl (3 mg) | yes |
| Control pellet | — | 100 μl (1 mg) | 300 μl (3 mg) | yes |
| bFGF alone | 1 μg | — | — | — |

All compositions, including controls, were injected or implanted into the back subcutaneous tissue of rats. It was known that bFGF promotes neovascularization. Seven days after administration, each rat was evaluated for neovascularization at the area peripheral to the site of injection or implantation. It was discovered that the pellet containing bFGF induced substantial neovascularization.

To distinguish newly synthesized blood vessels from pre-existing ones, lipid microspheres were injected into mice just superficial to the pre-existing capillary bed early in the experiment (at day 0). At the end of the experiment, the neovascular capillaries, if any, would reside superficial to the microspheres. Thus, new capillaries were easily identified by seeing whether there was vascularization above (superficial to) the microspheres at the end of the experiment. Significant neovascularization was observed in the rat receiving the pellet containing bFGF, but no neovascularization was observed in rats receiving bFGF alone nor a control pellet without bFGF.

### OTHER EMBODIMENTS

It is to be understood that while the invention has been described in conjunction with the detailed description thereof, the foregoing description is intended to illustrate and not limit the scope of the invention, which is defined by the appended claims. Other aspects, advantages, and modifications are within the scope of this invention.

What is claimed is:

1. A composition providing sustained release of a drug, the composition comprising a mucopolysaccharide, a carrier protein, and a drug.

2. The composition of claim 1, wherein the composition consists of the mucopolysaccharide, the carrier protein, the drug, and one or more pharmaceutically acceptable additives.

3. The composition of claim 1, wherein the ratio of the total mass of mucopolysaccharide in the composition to the total mass of carrier protein in the composition is about 1:1 to 1:20.

4. The composition of claim 1, wherein the mucopolysaccharide is chondroitin sulfate or hyaluronate.

5. The composition of claim 1, wherein the carrier protein is a γ-globulin, albumin, fibrinogen, histone, protamine, gelatin, or collagen.

6. The composition of claim 1, wherein the carrier protein is a γ-globulin.

7. The composition of claim 1, wherein the carrier protein is an albumin.

8. The composition of claim 1, wherein the drug is a protein drug.

9. The composition of claim 8, wherein the protein drug is an erythropoietin, granulocyte colony stimulating factor, granulocyte macrophage colony stimulating factor, thrombopoietin, interferon-α, interferon-β, interferon-γ, urokinase, tissue plasminogen activator, interleukin-11, fibroblast growth factor, epidermal growth factor, growth hormone, brain-derived neurotrophic factor, nerve growth factor, leptin, neurotrophin-3, superoxide dismutase, antibody, calcitonin, insulin, or parathyroid hormone.

10. The composition of claim 1, wherein the composition contains about 0.1 to 50% by weight the mucopolysaccharide.

11. The composition of claim 1, wherein the composition contains about 0.1 to 2% by weight the drug.

12. A method of producing a sustained release drug composition, the method comprising
providing a precipitating solution containing a mucopolysaccharide, a carrier protein, and a drug;
lowering the pH of the precipitating solution to a level sufficient to form an insoluble product comprising the mucopolysaccharide, the carrier protein, and the drug; and
collecting from the precipitating solution the insoluble product.

13. The method of claim 12, wherein the insoluble product consists of the mucopolysaccharide, the carrier protein, the drug, and one or more pharmaceutically acceptable additives.

14. The method of claim 12, wherein the ratio of the total mass of mucopolysaccharide in the insoluble product to the total mass of carrier protein in the insoluble product is about 1:1 to 1:20.

15. The method of claim 12, wherein the mucopolysaccharide is chondroitin sulfate or hyaluronate.

16. The method of claim 12, wherein the carrier protein is a γ-globulin, albumin, fibrinogen, histone, protamine, gelatin, or collagen.

17. The method of claim 12, wherein the carrier protein is a γ-globulin.

18. The method of claim 12, wherein the carrier protein is an albumin.

19. The method of claim 12, wherein the drug is a protein drug.

20. The method of claim 12, wherein the protein drug is an erythropoietin, granulocyte colony stimulating factor, granulocyte-macrophage colony stimulating factor, thrombopoietin, interferon-α, interferon-β, interferon-γ, urokinase, tissue plasminogen activator, interleukin-11, fibroblast growth factor, epidermal growth factor, growth hormone, brain-derived neurotrophic factor, nerve growth factor, leptin, neurotrophin-3, superoxide dismutase, antibody, calcitonin, insulin, or parathyroid hormone.

21. The method of claim 12, wherein the pH of the solution is about 7 or above before the lowering step.

22. The method of claim 12, wherein the pH of the solution is lowered to about 2 to 4 in the lowering step.

23. The method of claim 12, further comprising, prior to the providing step, mixing a first solution containing the carrier protein and the drug with a second solution containing the mucopolysaccharide to produce the precipitating solution.

24. The method of claim 12, wherein the precipitating solution contains zinc or calcium ions.

25. The method of claim 12, further comprising
suspending the insoluble product in a preparatory solution having a pH of about 6 to 8 to form a mixture; and
lyophilizing the mixture to obtain a solid product.

26. A composition providing sustained release of a drug, the composition comprising a mucopolysaccharide and a protein drug.

27. The composition of claim 26, wherein the composition consists of the mucopolysaccharide, the protein drug, and one or more pharmaceutically acceptable additives.

28. The composition of claim 26, wherein the mucopolysaccharide is chondroitin sulfate or hyaluronate.

29. The composition of claim 26, wherein the protein drug is an erythropoietin, granulocyte colony stimulating factor, granulocyte-macrophage colony stimulating factor, thrombopoietin, interferon-α, interferon-β, interferon-γ, urokinase, tissue plasminogen activator, interleukin-11, fibroblast growth factor, epidermal growth factor, growth hormone, brain-derived neurotrophic factor, nerve growth factor, leptin, neurotrophin-3, superoxide dismutase, antibody, calcitonin, insulin, or parathyroid hormone.

30. The composition of claim 26, wherein the composition contains about 0.1 to 50% by weight the mucopolysaccharide.

31. The composition of claim 26, wherein the composition contains about 0.1 to 50% by weight the protein drug.

32. A method of producing a sustained release drug composition, the method comprising
providing a precipitating solution containing a mucopolysaccharide and a protein drug;
lowering the pH of the precipitating solution to a level sufficient to form an insoluble product comprising the mucopolysaccharide and the protein drug; and
collecting from the precipitating solution the insoluble product.

33. The method of claim 32, wherein the insoluble product consists of the mucopolysaccharide, the protein drug, and one or more pharmaceutically acceptable additives.

34. The method of claim 32, wherein the mucopolysaccharide is chondroitin sulfate or hyaluronate.

35. The method of claim 32, wherein the protein drug is an erythropoietin, granulocyte colony stimulating factor, granulocyte-macrophage colony stimulating factor, thrombopoietin, interferon-α, interferon-β, interferon-γ, urokinase, tissue plasminogen activator, interleukin-11, fibroblast growth factor, epidermal growth factor, growth hormone, brain-derived neurotrophic factor, nerve growth factor, leptin, neurotrophin-3, superoxide dismutase, antibody, calcitonin, insulin, or parathyroid hormone.

36. The method of claim 32, wherein the pH of the solution is about 7 or above before the lowering step.

37. The method of claim 32, wherein the pH of the solution is lowered to about 2 to 4 in the lowering step.

38. The method of claim 32, further comprising, prior to the providing step, mixing a first solution containing the protein drug with a second solution containing the mucopolysaccharide to produce the precipitating solution.

39. The method of claim 32, wherein the precipitating solution contains zinc or calcium ions.

40. The method of claim 32, wherein the insoluble product contains about 0.1 to 50% by weight the mucopolysaccharide.

41. The method of claim 32, wherein the insoluble product contains about 0.1 to 50% by weight the protein drug.

42. The method of claim 32, further comprising
suspending the insoluble product in a preparatory solution having a pH of about 6 to 8 to form a mixture; and
lyophilizing the mixture to obtain a solid product.

43. A method of delivering a drug to a subject, the method comprising introducing the composition of claim 1 into the subject.

44. The method of claim 43, wherein the composition is introduced subcutaneously or intramuscularly into the subject.

45. A method of delivering a drug to a subject, the method comprising introducing the composition of claim 26 into the subject.

46. The method of claim 45, wherein the composition is introduced subcutaneously or intramuscularly into the subject.
